(19) United States
(12) Patent Application Publication
(10) Pub. No.: US 2013/0023998 A1
(43) Pub. Date: Jan. 24, 2013

(54) SHOULDER INTER-SPACER COMPONENT AND SURGICAL METHOD OF IMPLANTATION

(71) Applicant: Rajiv D. Pandya, Atlanta, GA (US)

(72) Inventor: Rajiv D. Pandya, Atlanta, GA (US)

(21) Appl. No.: 13/187,632

(22) Filed: Jul. 21, 2011

(51) Int. Cl. A61F 2/40

(52) U.S. Cl. 623/19.11

(57) ABSTRACT

A shoulder inter-spacer implant has a first outer surface having a contoured profile simulating a glenoid surface and a second inner surface for resting on an outer surface of a prepared glenoid. The second inner surface has a flat or contoured profile void of any pegs, keels or bone interlocking projections so that the second inner surface is profiled to rest on the surface of the prepared glenoid when implanted free of any direct anchoring or cementing to the bone structure. In this fashion the inter-spacer is free to move or slide on the prepared glenoid. The inter-spacer may include one or more soft tissue attachment locations to which a tendon, cartilage, ligament or other soft tissue can be secured, the inter-spacer being otherwise free floating relative to the prepared glenoid.

### FIELD OF THE INVENTION

The present invention relates to the field of shoulder repair or replacement, more particularly to an improved implant device and method for glenoid repair or replacement.

### BACKGROUND OF THE INVENTION

The invention provides an improved glenoid shoulder implant.

Shoulder replacement surgery is currently used to treat patients having worn or damaged shoulder joints. Glenoid shoulder socket implants are typically made completely from high or ultra high density polyethylene and affixed to the cortical bone using bone cement, polymethylmethacrylate. Some glenoid implants have a metal base plate with a polyethylene insert. Current glenoid implants use either a keel or multiple pegs on the back or inner surface of the prosthetic glenoid implant to anchor and secure the glenoid implant rigidly fixed to the inside the glenoid vault.

In US patent publication 2011/0112648 entitled “Methods For Less Invasive Glenoid Replacement” a litany of problems of the current practice of glenoid repair is recited. It was reported that, “Keeled and pegged glenoid implants suffer from several disadvantages, which limit their lifespan once implanted and reduce the number of indications for which they can be used when the age of the patient is a factor. For example, the glenoid implants can loosen due to poor fixation within the bone, and they are prone to wear and fatigue failure of the polyethylene due to adhesion, abrasion, and shear stress. Because of these deficiencies, surgeons hesitate to perform glenoid replacement surgery on young or middle aged patients with glenoid articular cartilage injuries or damage due to early arthritis for fear that the implant may not last more than 10-15 years in the body, thus subjecting the patient to the possibility of two or more surgeries during the lifetime of the patient to preserve the function and pain-free state of the joint. Finally, current glenoid implants with a long keel or pegs are sometimes contraindicated in patients with significant glenoid bone loss. As arthritis progresses, the humeral head can wear medially and destroy the foundation of glenoid bone. In these cases, the glenoid vault can be significantly reduced in volume and depth. Thus, a typical keel or peg design can broach the glenoid vault and injure the suprascapular nerve along the suprascapular notch or spinoglenoid notch with resultant denervation injury to the rotator cuff muscles. Broaching through the glenoid vault can also fracture the body of the scapula and cause early implant loosening.”

The inventors in US 2011/0112648 went on to state, “There are also several disadvantages associated with current glenoid replacement surgical techniques. Current techniques require extensive shoulder exposure with capsular releases in order to fully expose the glenoid surface circumferentially. Since the axillary nerve is located within 1 cm of the inferior capsule, there is potential risk of axillary nerve injury with resultant denervation injury to the deltoid muscle when these releases are performed. However, use of the current keeled or pegged glenoid implants requires this extensive glenoid exposure for proper fitting and placement. Current glenoid replacement surgery also requires a long skin incision and extensive soft tissue stripping in order to fully expose the glenoid circumferentially, which produces a cosmetically unappealing scar. Finally, current glenoid replacement surgical techniques require advanced surgical training and expertise within the specialty of shoulder surgery, yet the majority of shoulder implants performed in the U.S. every year are performed by orthopedic surgeons who do not have advanced training in the subspecialty of shoulder surgery. Therefore, many surgeons have difficulty preparing the glenoid site for a total shoulder replacement using the current techniques. Because there are more than 20,000 shoulder arthoplasty surgeries performed per year, many U.S. patients incur a risk of continued pain and disability, neuromuscular injuries, or failed shoulder prostheses requiring revision surgery. Thus, there remains a need for an improved glenoid implant and improved methods for performing replacement shoulder surgery.”

Their solution to these listed deficiencies was stated in US 2011/0112648 to be: a glenoid implant itself includes a (1) body portion having (i) a smooth concave lateral articulating surface facing away from the scapula, which is adapted to be engaged by a convex surface of a humeral component, and (ii) an opposing surface on the medial side intended to be positioned within a cavity reamed in the glenoid. In their embodiment, the glenoid implant also includes (2) a short peg on the medial side extending centrally outward along an axis from a convex or flat backside (medial) surface of the glenoid implant. In the preferred embodiment, the short peg of the glenoid implant is less than about 10 mm long, more preferably about 8 mm or less in length, even more preferably about 5 mm or less in length. Alternatively, the glenoid implant has multiple pegs, each of which can be the same length or different lengths, e.g., less than about 8 mm or less in length, more preferably about 5 mm or less in length. In another embodiment, at least one of the pegs is between about 5 mm and about 8 mm in length and the remaining pegs are less than about 8 mm in length.

This prior art glenoid implant was secured to the glenoid using cement fixation or press fit technique. In yet another preferred embodiment, the glenoid implant is further secured to the glenoid using screws in press fit designs.

In fact the teaching of US 2011/0112648 are simply a minor adjustment in the typical or current prior art techniques. Their implant device still requires the anchoring and permanent fixation of the implant to the glenoid vault and furthermore requires bone removal to accomplish this albeit less than required with the older styled elongated pegs and keel. Their shortened pegs still require bone cavities to be created and the optional method of screwing the implant to the bone and the use of bone cementing all of which are well known and accepted practices.

Nevertheless, this prior art document does reflect an accurate portrayal of the problems occurring in this type of surgery.

The present invention as described below solves several of the problems in a completely unique way heretofore never attempted.

### SUMMARY OF THE INVENTION

A shoulder inter-spacer implant has a first outer surface having a contoured profile simulating a glenoid surface and a second inner surface for resting on an outer surface of a prepared glenoid. The second inner surface has a flat or contoured profile void of any pegs, keels or bone interlocking projections so that the second inner surface is profiled to rest on the surface of the prepared glenoid when implanted free of any direct anchoring or cementing to the bone structure. The second inner surface may optionally include a roughened or textured surface to facilitate positioning and limiting movement, but does not include any rigid fixation to the glenoid such as cement or screws. In this fashion the inter-spacer is free to move or slide on the prepared glenoid. The inter-spacer may include one or more soft tissue attachment locations to which a tendon, cartilage, ligament or other soft tissue can be secured, the inter-spacer being otherwise free floating relative to the prepared glenoid.

Use of this shoulder inter-spacer is best summarized by the following method. A method of treating a patients shoulder has the steps of a) identifying a patient having a weakened or damaged glenoid surface needing repair or replacement; b) surgically exposing the shoulder having a weakened or damaged glenoid; c) inserting a shoulder inter-spacer having a glenoid surface over the weakened or damaged glenoid surface or a surgically prepared surface of the glenoid wherein the spacer is free floating relative to the glenoid bone structure and is positioned between the humeral head and the glenoid being repaired or replaced; and d) surgically closing the exposed shoulder.

The method may further have the step of attaching at least one of a ligament, cartilage, tendon or other soft tissue to the shoulder inter-spacer to assist in positioning the free floating spacer between the humeral head and the repaired glenoid. The method may further include surface preparation of the glenoid by removing the weakened or damaged glenoid surface and providing a generally flat or slightly contoured surface onto which the inter-spacer is positioned, the implant rests on prepared glenoid surface in the absence of mechanical protrusions, pegs or a keel and the preparation of the glenoid is accomplished in the absence of bone tissue removal to form internal fixation cavities. The inter-spacer not being rigidly anchored or otherwise secured to the bone structure by mechanical means including screws or fasteners. The otherwise free floating inter-spacer can have non-rigid soft tissue attached to a peripheral or an exterior surface of the inter-spacer to facilitate positioning the inter-spacer between the humeral head and the surgically prepared glenoid prior to closing the exposed shoulder.

### BRIEF DESCRIPTION OF THE DRAWINGS

The invention will be described by way of example and with reference to the accompanying drawings in which:

FIG. 1 is a front perspective view of an exemplary circular shoulder inter-spacer for use as a glenoid implant of the present invention.

FIG. 2 is a rear perspective view of the shoulder inter-spacer of FIG. 1.

FIG. 2A is a view taken from FIG. 2 showing an optional textured or roughened second surface.

FIG. 3 is a cross sectional view of the shoulder inter-spacer taken along lines 3-3 of FIG. 1.

FIG. 4 is a plan view of a normal shoulder showing the relative position of the humeral head and the glenoid surface.

FIG. 5 is a view of a repaired shoulder with the inter-spacer shown positioned between the humeral head and the surgically prepared glenoid surface.

FIG. 6 is a view of the shoulder inter-spacer with soft tissue attached.

### DETAILED DESCRIPTION OF THE INVENTION

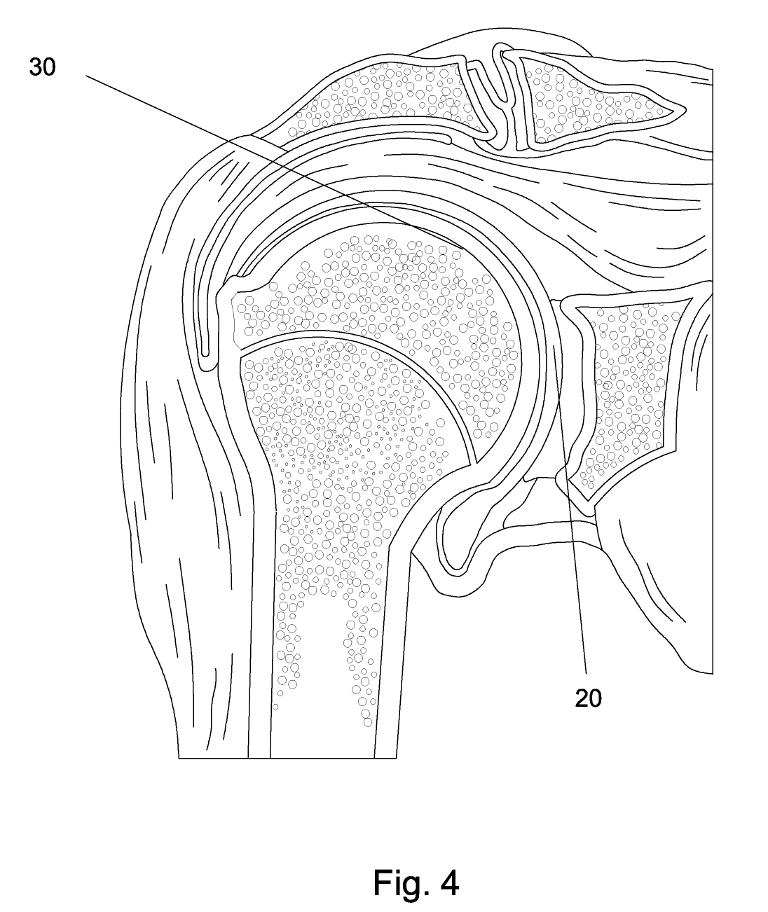

With reference to FIG. 4 a normal shoulder is shown. Often the humeral bone 30 or the glenoid 20 can become worn or damaged and a surgical repair or replacement is required.

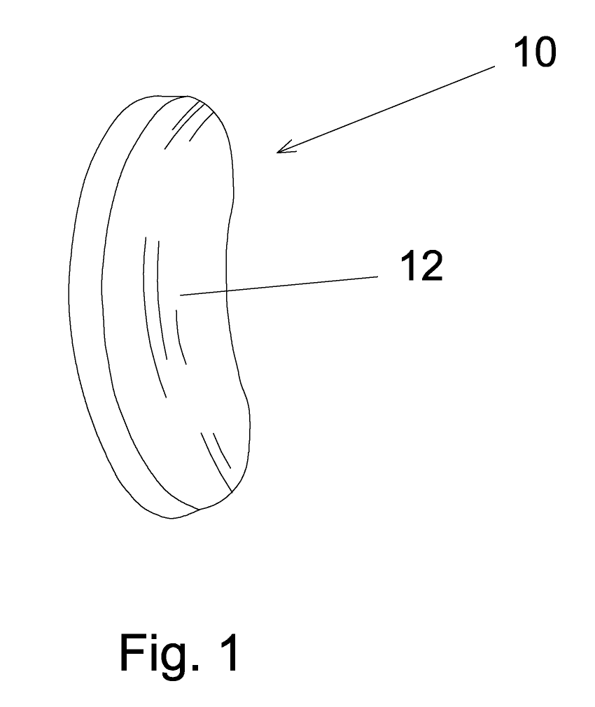

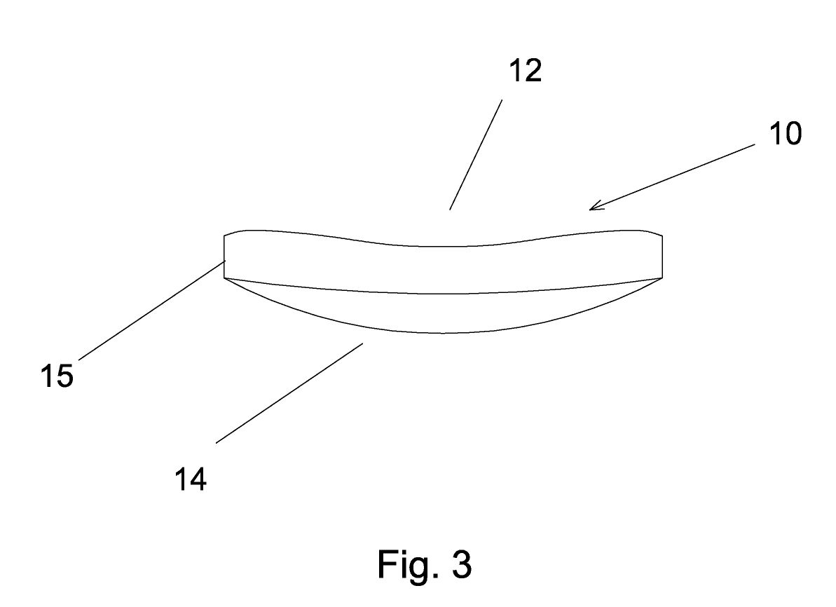

With reference to FIGS. 1-3, there is shown an exemplary shoulder inter-spacer 10 of the present invention. As illustrated, the shoulder inter-spacer 10 has a generally oval or circular disk like shape with a first or frontal surface 12 having a shallow concavity formed by one or more radii of curvature. The front surface 12 is shaped to duplicate or replicate the surface profile of the glenoid fossia. The shoulder inter-spacer 10 exhibiting this first frontal surface 12 formed to replicate the glenoid surface enables the inter-spacer 10 to be used in a glenoid replacement or repair procedure.

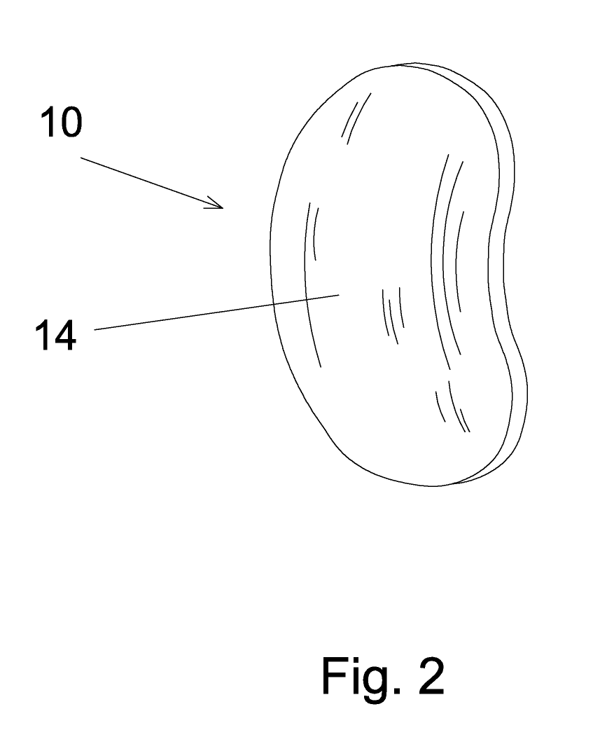

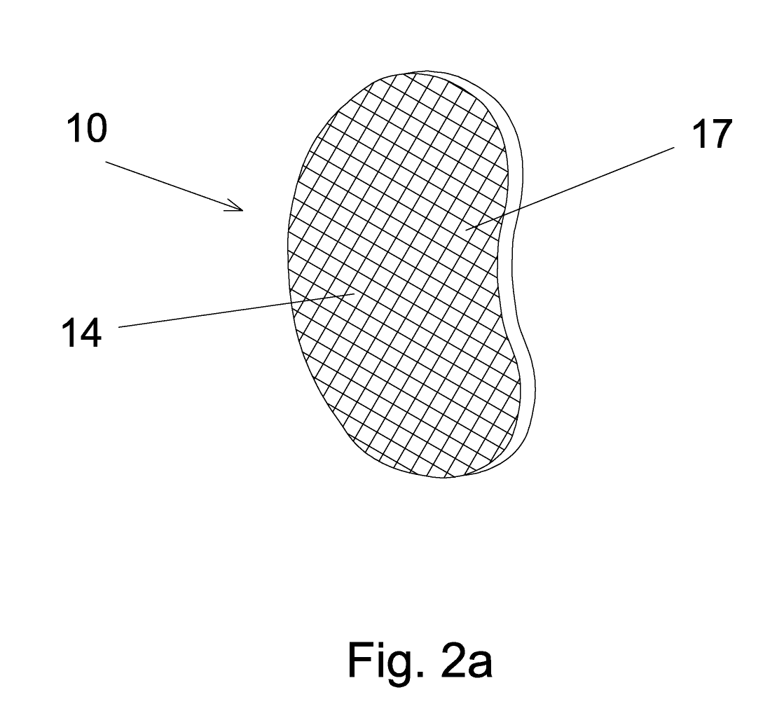

With reference to FIG. 2, the rear perspective of the shoulder inter-spacer 10 is illustrated showing a second or rear surface 14. The second or rear surface as shown has a generally flat or slightly contoured surface that is devoid of any projecting pegs or keels. This second surface 14 is made to rest on the exterior outer surface of a glenoid which has been prepared to accept the inter-spacer 10. As shown in FIG. 2A, the second surface 14 may optionally include a roughened or textured surface 17 to facilitate positioning and limiting slipping movement, but does not include any rigid fixation to the glenoid surface such as pegs, keels, cement or fixation screws.

As further illustrated in the cross sectional view of the inter-spacer 10, the first frontal surface 12 more clearly shows the hollow concavity that mimics or replicates the glenoid surface. When implanted, the inter-spacer first or frontal surface 12 is in contact with the humeral head. The thickness of the inter-spacer 10 can vary slightly from a minimum at the center to a maximum radially outwardly as shown. The profile of the frontal surface 12 is a shallow curvature formed by two or more radii approximating the curvature of the normal glenoid.

As illustrated, the inter-spacer 10 is preferably made of a synthetic thermoplastic such as a high density polyethylene. What is a most distinguished feature of the inter-spacer 10 is the second or rear surface 14 has no fixing or anchoring features projecting from it. In fact, such features are avoided and not desirable. Additionally, there are no holes or apertures in the shoulder inter-spacer 10 for allowing fasteners or screws to be used to anchor or fix to the underlying bone tissue. Remarkably, the use of such anchoring or fixing devices to secure the inter-spacer 10 to the bone tissue is not needed and more importantly are undesirable.

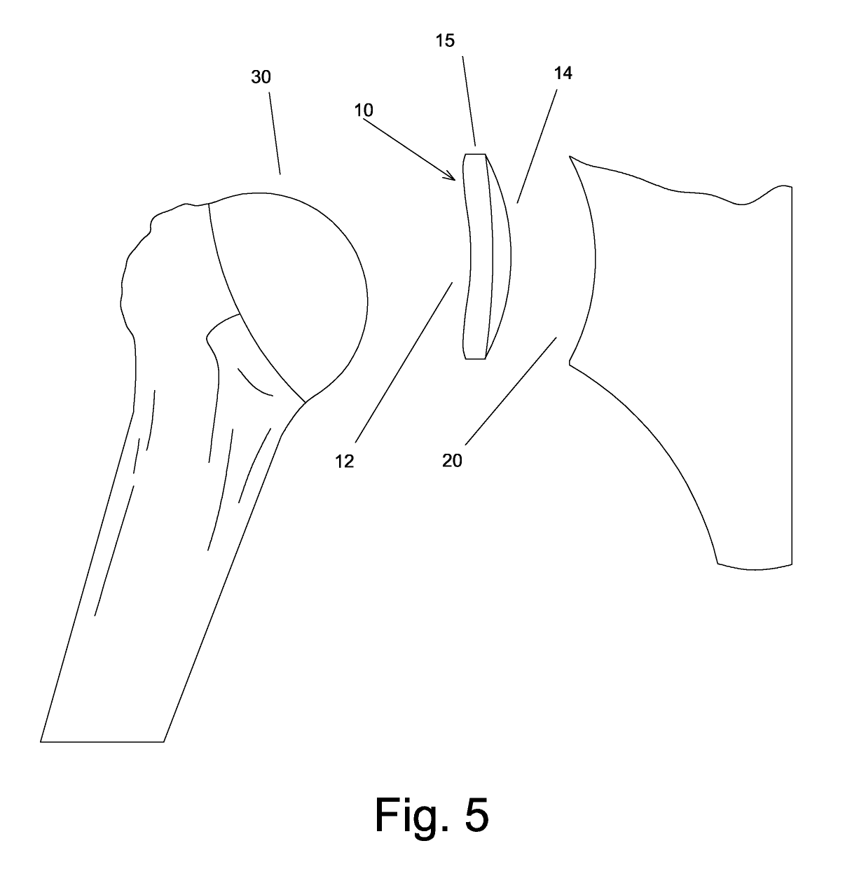

The inter-spacer 10 is designed to rest on and essentially be free floating relative to the glenoid cavity on which it rests. Remarkably, no bone cement is needed either. The concept of the shoulder inter-spacer 10 relies on the fact that the inter-spacer is allowed to move slightly between the prepared glenoid surface 20 and the humeral head 30 which effectively holds the inter-spacer 10 between these two anatomical features as shown in FIG. 5.

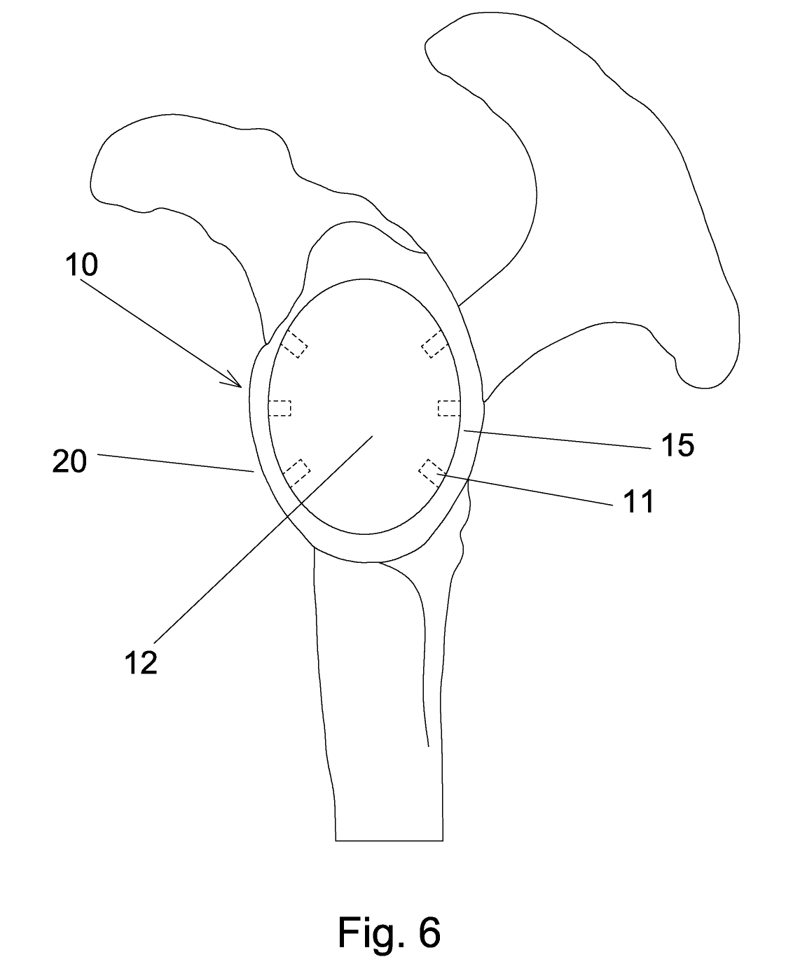

In FIG. 6, the shoulder inter-spacer 10 optionally can have one or more holes 11 on the side or peripheral edge 15 of the inter-spacer 10. These holes 11 are provided to allow soft tissue such as cartilage, ligaments or tendons to be attached. These soft tissue attachments provide an optional facilitation of holding the inter-spacer 10 in relative position without impeding or over restricting the free floating ability of the inter-spacer 10 relative to the adjacent glenoid surface 20 on which the second or rear surface 14 rests. While this attached soft tissue may be attached to the bone structure, it can stretch and move enough to let the inter-spacer 10 float when implanted. Preferably the holes 11 may be threaded to accept a fixation screw of fastener.

What is unique and remarkable about the shoulder inter-spacer 10 is that it is deliberately made to be loose relative to the underlying bone structure. Accordingly, unlike fixed and cemented implants which are considered surgical failures when the implants become loose because the underlying bone tissue has been weakened by the removal of large amounts of bone tissue and a loose implant with projections such as pegs and keels can break the weak bone as it moves, the present invention avoids these complications. When the present device is used very little bone tissue needs to be removed to create anchoring cavities. Instead the patient's bone structure is substantially left intact and only the damaged glenoid surface bone tissue needs to be removed to form a substantially flat or slightly contoured surface like a table top for the shoulder implant to float on. Remarkably, this procedure is very reliable in the sense that a list of unnecessary bone preparation procedures are avoided, risk of complications are reduced due to bone fractures and the entire complexity of cementing a peg or keel in a fluid filled bone cavity carved into the patients bone is avoided. Secondarily, the surgeons skill level while still extremely important is less critical because some of the most difficult and delicate aspects of surgically preparing the bone tissue are eliminated.

The patient having an inter-spacer 10 implanted as described above will also experience far less pain and damage and the healing prospects and recovery will be improved due to this simplified procedure.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treating a patient's shoulder comprising:
identifying a patient having a weakened or damaged glenoid surface needing repair or replacement;
surgically exposing the shoulder having a weakened or damaged glenoid;
inserting a shoulder inter-spacer having a glenoid surface, either directly over the weakened or damaged glenoid surface or a surgically prepared surface of the glenoid wherein the spacer is free floating relative to the glenoid bone structure and is positioned between the humeral head and the glenoid being repaired or replaced; and
surgically closing the exposed shoulder.

2. The method of treating a patient's shoulder of claim 1 further comprises the step of:
attaching at least one of ligament, tendon, cartilage or other soft tissue to the shoulder inter-spacer to assist in positioning the free floating spacer between the humeral head and the repaired glenoid.

3. The method of treating a patient's shoulder of claim 1 wherein the step of surgically preparing the damaged or weakened glenoid surface includes the step of:
surface preparing the glenoid by removing the weakened or damaged glenoid surface and providing a generally flat or slightly contoured surface onto which the shoulder inter-spacer is positioned.

4. The method of treating a patient's shoulder of claim 3 wherein the shoulder inter-spacer rests on the prepared glenoid surface in the absence of mechanical protrusions, pegs or a keel and the step of surgically preparing the glenoid is accomplished in the absence of bone tissue removal to form internal fixation cavities.

5. The method of treating a patient's shoulder of claim 4 wherein the step of inserting the inter-spacer is accomplished wherein the inter-spacer is placed onto the prepared glenoid surface and not anchored or otherwise secured to the bone structure by mechanical screws or fasteners.

6. The method of treating a patient's shoulder of claim 5 wherein the method further comprises the step of:
attaching soft tissue to the peripheral or an exterior surface of the shoulder inter-spacer to facilitate positioning the inter-spacer between the humeral head and the surgically prepared glenoid.

7. A shoulder inter-spacer implant comprises:
a spacer having a first outer surface having contoured profile simulating a glenoid surface and a second inner surface for resting on the outer surface of a prepared glenoid.

8. A shoulder inter-spacer implant of claim 7 wherein the second inner surface has a flat or contoured profile.

9. A shoulder inter-spacer implant of claim 8 wherein the inner surface flat or contoured profile of the inner surface is void of any pegs, keels or projections, the inner surface being profiled to rest on the surface of the prepared glenoid when implanted.

10. A shoulder inter-spacer implant of claim 9 wherein the second inner surface is smooth, roughened or textured.

11. A shoulder inter-spacer implant of claim 9 wherein the inter-spacer may include one or more soft tissue attachment locations to which a tendon, cartilage, ligament or other soft tissue can be secured, the inter-spacer being otherwise free floating relative to the prepared glenoid.
